(19) United States
(12) Patent Application Publication
(10) Pub. No.: US 2025/0229075 A1
(43) Pub. Date: Jul. 17, 2025

(54) HANGING VALVE NEEDLE FREE CONNECTOR

(71) Applicant: CareFusion 303, Inc., San Diego, CA (US)

(72) Inventors: Ryan CALLAHAN, Long Beach, CA (US); Jason Andrew WINE, Brea, CA (US); Siddarth SHEVGOOR, Mission Viejo, CA (US)

(21) Appl. No.: 19/002,055

(22) Filed: Dec. 26, 2024

Related U.S. Application Data
(60) Provisional application No. 63/621,948, filed on Jan. 17, 2024.

(51) Int. Cl. A61M 39/26; A61M 39/10

(52) U.S. Cl. CPC A61M 39/26; A61M 2039/1083; A61M 2039/1088

(57) ABSTRACT

Aspects of the present disclosure provide a fluid connector assembly, comprising a housing, comprising an upper housing including a female luer port, a lower housing including a male luer port and a cannula flow channel passing through the male luer port, and an internal volume fluidly connected to a first end and a second end and an elastomeric valve disposed in the internal volume, the elastomeric valve comprising a cavity, wherein when the male luer port is inserted into the female luer port of the upper housing, the elastomeric valve is configured to be stretched and pushed downwards, allowing a tip of the male luer port to interface with the cannula flow channel and for fluid to flow through the fluid connector assembly.

### CROSS REFERENCE TO RELATED APPLICATION

This application claims the benefit of priority under 35 U.S.C. § 119 to U.S. Provisional Patent Application Ser. No. 63/621,948, entitled “HANGING VALVE NEEDLE FREE CONNECTOR”, filed on Jan. 17, 2024, the entire contents of which is incorporated herein by reference.

### TECHNICAL FIELD

The present disclosure generally relates to medical infusion fluid lines connectors, and more particularly to a needle free connector (or luer activated device) utilizing a hanging elastomeric valve.

### BACKGROUND

Needle-free connectors, including hanging valve needle-free connectors, offer a solution for providing medical fluid to patients. In an exemplary embodiment, a needle-free connector assembly promotes fluid transmission between a medical fluid supply and a catheter line. The medical fluid supply and the catheter line are secured to respective luers that are secured to a connector body. To transmit fluid, the luer connected to the medical supply is inserted into the connector body and overlaps a central post of the luer connected to the catheter line.

Typical compression type luer activated devices (“LAD”) utilize a valve that has to be compressed to activate the device. These valves are large and require a lot of material. Also, extra space is needed for the valve to expand as it is compressed. Furthermore, typical LADs require an additional seal at the base of the device to prevent fluid leakage in the device.

### SUMMARY

In accordance with at least some embodiments disclosed herein is the realization that components used with neutral fluid displacement, such as a luer and a post, can break when overlapping and coming into contact with each other, which can result in medical fluid leakage and reduced medical delivery to a patient. When a broken component is located in a connector body, it may not be readily present to a medical professional that the component is broken, leading to delayed response times.

The present disclosure generally relates to luer connections, and more particularly to a hanging valve needle free connector that utilizes a valve that is stretched during activation. The prior art uses a valve that is compressed instead of stretched during activation. The hanging valve design where the valve is stretched during activation requires less material as the valve does not need to act as a compressible spring and less extra space inside the connector as it does not need to expand downward and outward when activated. The hanging valve also acts as a secondary seal and reduces interstitial space.

Accordingly, aspects of the present disclosure provide a fluid connector assembly comprising a housing, comprising an upper housing including a female luer port, a lower housing including a male luer port and a cannula flow channel passing through the male luer port, and an internal volume fluidly connected to a first end and a second end, and an elastomeric valve disposed in the internal volume, the elastomeric valve comprising a cavity, wherein when the male luer port is inserted into the female luer port of the upper housing, the elastomeric valve is configured to be stretched and pushed downwards, allowing a tip of the male luer port to interface with the cannula flow channel and for fluid to flow through the fluid connector assembly.

In some embodiments, the male luer port displaces the elastomeric valve and causes a fluid entering the upper housing to pass through the cannula flow channel. In some embodiments, the elastomeric valve seals the female luer port and cannula flow channel when the male luer port is not inserted in the female luer port. In some embodiments, the elastomeric valve is constrained between the upper housing and lower housing. In some embodiments, the elastomeric valve stretches when it is activated. In some embodiments, the elastomeric valve acts as a seal to prevent leakage in the fluid connector assembly in both an active and a resting state. In some embodiments, the elastomeric valve is configured to get smaller and thin out as the elastomeric valve is stretched. In some embodiments, the elastomeric valve does not expand downwards or outwards when activated.

Some instances of the present disclosure provide a method or regulating an intravenous fluid, by a fluid connector assembly, comprising providing a housing, the housing comprising an in internal volume, receiving, at the housing, an elastomeric valve in the internal volume, receiving, at a lower housing, a male luer port, the male luer port comprising a post and an opening formed in the post, receiving, at an upper housing, a female luer port, stretching, based on receiving the male luer port, the elastomeric valve and forming, based on the stretching of the elastomeric valve, a seal that causes the intravenous fluid that enters the fluid connector assembly to pass through the opening. In some embodiments, stretching the elastomeric valve comprises stretching the elastomeric valve from a first dimension to a second dimension greater than the first dimension.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology as claimed. It is also to be understood that other aspects may be utilized, and changes may be made without departing from the scope of the subject technology.

### BRIEF DESCRIPTION OF THE DRAWINGS

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

FIG. 1 illustrates an IV administration set coupled to a patient, in accordance with aspects of the present disclosure.

FIG. 2 is a cross sectional view of a hanging valve needle free connector in accordance with some embodiments of the present disclosure.

FIG. 3 is a flow chart illustrating a method for regulating an intravenous fluid using a hanging valve needle free connector in accordance with embodiment of the present disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions may be provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

In accordance with some embodiments, the present disclosure includes various features and advantages of maintaining separation between a post and luer, thus minimizing the likelihood of damaging the post and/or the luer.

The present description relates in general to infusion fluid lines connectors, and more particularly, for example and without limitation, to more particularly to needle free connector utilizing a hanging elastomeric valve.

In some embodiments, the needless connector is used in an IV administration set having a structure for coupling with an IV bag and/or a drip chamber. The structure for the IV bag can be formed as an arm, hook, clamp, or another mechanism configured to suspend the IV bag.

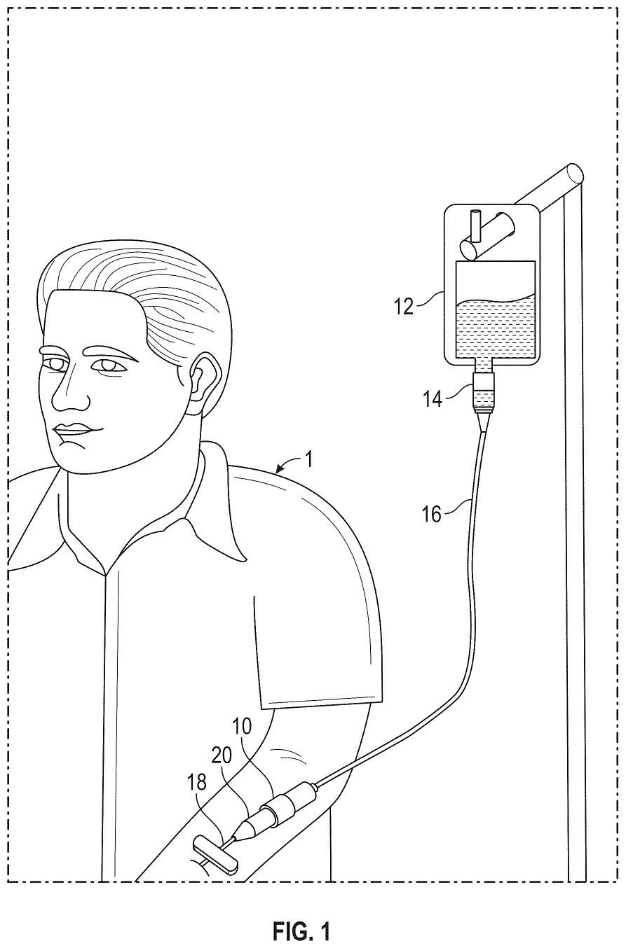

Referring to FIGS. 1, an embodiment of an IV administration set 2 is coupled to a patient 1. The IV administration set 2 may include a fluid connector assembly 100. The IV administration set includes an IV bag 12 and a drip chamber 14 fluidly coupled together by IV tubing 16 extending therebetween. The IV tubing 16 is intravenously coupled to the patient 1 through a catheter 18, and the catheter preferably includes one or more adapters 10 or connectors 20.

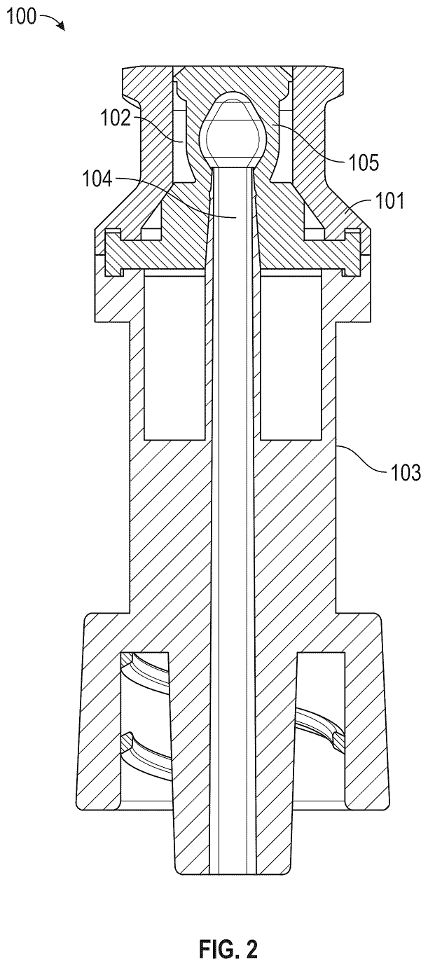

Referring to FIG. 2, an embodiment of a fluid connector assembly 100 is shown, the fluid connector assembly 100 comprising a housing, the housing comprising an upper housing 101 including a female luer port 102, a lower housing 103 including a male luer port 104 and a cannula flow channel passing through the male luer port 104, an internal volume fluidly connected to a first end and a second end and an elastomeric valve 105 disposed in the internal volume, the elastomeric valve 105 comprising a cavity, wherein when the male luer port 104 is inserted into the female luer port 102 of the upper housing 101, the elastomeric valve 105 is configured to be stretched and pushed downwards, allowing a tip of the male luer port 104 to interface with the cannula flow channel and for fluid to flow through the fluid connector assembly 100.

In some embodiments, the male luer port 104 displaces the elastomeric valve 105 and causes a fluid entering the upper housing 101 to pass through the cannula flow channel. In some embodiments, the elastomeric valve 105 seals the female luer port 102 and cannula flow channel when the male luer port 104 is not inserted in the female luer port 102. In some embodiments, the elastomeric valve 105 is constrained between the upper housing 101 and lower housing 103. In some embodiments, the elastomeric valve 105 stretches when it is activated. In some embodiments, the elastomeric valve 105 acts as a seal to prevent leakage in the fluid connector assembly 100 in both an active and a resting state. In some embodiments, the elastomeric valve 105 is configured to get smaller and thin out as the elastomeric valve 105 is stretched. In some embodiments, the elastomeric valve 105 does not expand downwards or outwards when activated.

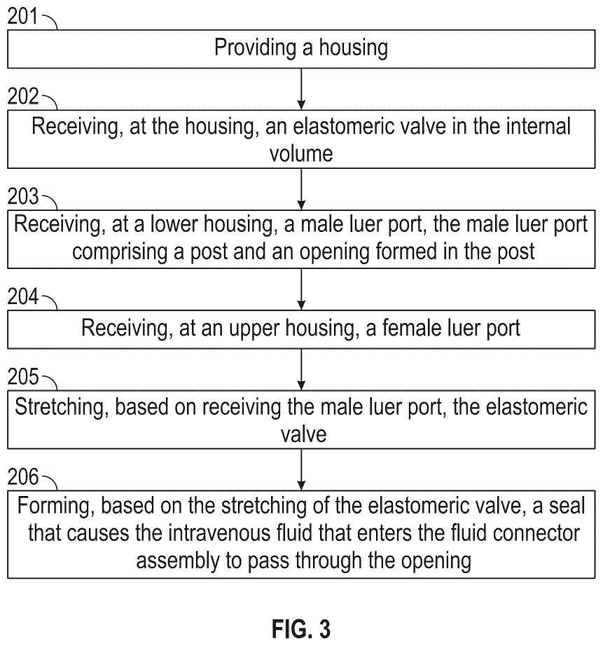

Referring to FIG. 3, embodiments of the present disclosure include a method for regulating an intravenous fluid, the method comprising, by a fluid connector assembly 100 providing 201 a housing, the housing comprising an in internal volume, receiving 202, at the housing, an elastomeric valve 105 in the internal volume, receiving 203, at a lower housing 103, a male luer port 104, the male luer port 104 comprising a post and an opening formed in the post, receiving 204, at an upper housing, a female luer port 102, stretching 205, based on receiving the male luer port 104, the elastomeric valve 105 and forming 206, based on the stretching of the elastomeric valve 105, a seal that causes the intravenous fluid that enters the fluid connector assembly 100 to pass through the opening. In some embodiments, stretching the elastomeric valve 105 comprises stretching the elastomeric valve 105 from a first dimension to a second dimension greater than the first dimension.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 9. The other clauses can be presented in a similar manner.

Clause 1: A fluid connector assembly, comprising a housing, comprising an upper housing including a female luer port; a lower housing including a male luer port and a cannula flow channel passing through the male luer port; and an internal volume fluidly connected to a first end and a second end; and an elastomeric valve disposed in the internal volume.

Clause 2: The fluid connector assembly of clause 1, wherein when the male luer port is inserted into the female luer port of the upper housing, the elastomeric valve is configured to be stretched and pushed downwards, allowing a tip of the male luer port to interface with the cannula flow channel and for fluid to flow through the fluid connector assembly.

Clause 3: The fluid connector assembly of clause 1, wherein the elastomeric valve comprising a cavity.

Clause 4: The fluid connector assembly of clause 1, wherein the male luer port displaces the elastomeric valve and causes a fluid entering the upper housing to pass through the cannula flow channel.

Clause 5: The fluid connector assembly of clause 1, wherein the elastomeric valve seals the female luer port and cannula flow channel when the male luer port is not inserted in the female luer port.

Clause 6: The fluid connector assembly of clause 1, wherein the elastomeric valve is constrained between the upper housing and lower housing.

Clause 7: The fluid connector assembly of clause 1, wherein the elastomeric valve stretches when it is activated.

Clause 8: The fluid connector assembly of clause 1, wherein the elastomeric valve acts as a seal to prevent leakage in the fluid connector assembly in both an active and a resting state.

Clause 9: The fluid connector assembly of clause 1, wherein the elastomeric valve is configured to get smaller and thin out as the elastomeric valve is stretched.

Clause 10: The fluid connector assembly of clause 1, wherein the elastomeric valve does not expand downwards when activated.

Clause 11: The fluid connector assembly of clause 1, wherein the elastomeric valve does not expand outwards when activated.

Clause 12: The fluid connector assembly of clause 1, wherein the elastomeric valve requires less material than a conventional hanging valve.

Clause 13: A method for regulating an intravenous fluid, the method comprising, by a fluid connector assembly: providing a housing, the housing comprising an in internal volume; receiving, at the housing, an elastomeric valve in the internal volume; receiving, at a lower housing, a male luer port, the male luer port comprising a post and an opening formed in the post; receiving, at an upper housing, a female luer port; and activating the elastomeric valve.

Clause 14: The method of clause 10, wherein activating the elastomeric valve includes stretching, based on receiving the male luer port, the elastomeric valve; and forming, based on the stretching of the elastomeric valve, a seal that causes the intravenous fluid that enters the fluid connector assembly to pass through the opening.

Clause 15: The method of clause 10, wherein when the elastomeric valve is activated, the elastomeric valve seals the female luer port and the opening.

Clause 16: The method of clause 10, wherein when the male luer is inserted into the female luer port, the elastomeric valve is stretched and pushed downwards, allowing for the intravenous fluid to flow through the fluid connector assembly.

Clause 17: The method of clause 10, wherein stretching the elastomeric valve comprises stretching the elastomeric valve from a first dimension to a second dimension greater than the first dimension.

Clause 18: The method of clause 10, wherein the elastomeric valve is configured to get smaller and thin out as the elastomeric valve is stretched.

Clause 19: The method of clause 10, wherein the elastomeric valve does not expand downwards when activated.

Clause 20: The method of clause 10, wherein the elastomeric valve does not expand outwards when activated.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. A fluid connector assembly, comprising:
a housing, comprising:
an upper housing including a female luer port;
a lower housing including a male luer port and a cannula flow channel passing through the male luer port; and
an internal volume fluidly connected to a first end and a second end; and
an elastomeric valve disposed in the internal volume.

2. The fluid connector assembly of claim 1, wherein when the male luer port is inserted into the female luer port of the upper housing, the elastomeric valve is configured to be stretched and pushed downwards, allowing a tip of the male luer port to interface with the cannula flow channel and for fluid to flow through the fluid connector assembly.

3. The fluid connector assembly of claim 1, wherein the elastomeric valve comprising a cavity.

4. The fluid connector assembly of claim 1, wherein the male luer port displaces the elastomeric valve and causes a fluid entering the upper housing to pass through the cannula flow channel.

5. The fluid connector assembly of claim 1, wherein the elastomeric valve seals the female luer port and cannula flow channel when the male luer port is not inserted in the female luer port.

6. The fluid connector assembly of claim 1, wherein the elastomeric valve is constrained between the upper housing and lower housing.

7. The fluid connector assembly of claim 1, wherein the elastomeric valve stretches when it is activated.

8. The fluid connector assembly of claim 1, wherein the elastomeric valve acts as a seal to prevent leakage in the fluid connector assembly in both an active and a resting state.

9. The fluid connector assembly of claim 1, wherein the elastomeric valve is configured to get smaller and thin out as the elastomeric valve is stretched.

10. The fluid connector assembly of claim 1, wherein the elastomeric valve does not expand downwards when activated.

11. The fluid connector assembly of claim 1, wherein the elastomeric valve does not expand outwards when activated.

12. The fluid connector assembly of claim 1, wherein the elastomeric valve requires less material than a conventional hanging valve.

13. A method for regulating an intravenous fluid, the method comprising, by a fluid connector assembly:
providing a housing, the housing comprising an in internal volume;
receiving, at the housing, an elastomeric valve in the internal volume;
receiving, at a lower housing, a male luer port, the male luer port comprising a post and an opening formed in the post;
receiving, at an upper housing, a female luer port; and
activating the elastomeric valve.

14. The method of claim 10, wherein activating the elastomeric valve includes stretching, based on receiving the male luer port, the elastomeric valve; and
forming, based on the stretching of the elastomeric valve, a seal that causes the intravenous fluid that enters the fluid connector assembly to pass through the opening.

15. The method of claim 10, wherein when the elastomeric valve is activated, the elastomeric valve seals the female luer port and the opening.

16. The method of claim 10, wherein when the male luer is inserted into the female luer port, the elastomeric valve is stretched and pushed downwards, allowing for the intravenous fluid to flow through the fluid connector assembly.

17. The method of claim 10, wherein stretching the elastomeric valve comprises stretching the elastomeric valve from a first dimension to a second dimension greater than the first dimension.

18. The method of claim 10, wherein the elastomeric valve is configured to get smaller and thin out as the elastomeric valve is stretched.

19. The method of claim 10, wherein the elastomeric valve does not expand downwards when activated.

20. The method of claim 10, wherein the elastomeric valve does not expand outwards when activated.
